(19) United States
(12) Patent Application Publication
(10) Pub. No.: US 2016/0287095 A1
(43) Pub. Date: Oct. 6, 2016

(54) BIOLOGICAL INFORMATION PROCESSING APPARATUS, BIOLOGICAL INFORMATION PROCESSING SYSTEM, BIOLOGICAL INFORMATION PROCESSING METHOD AND BIOLOGICAL INFORMATION PROCESSING PROGRAM

(71) Applicant: SEIKO EPSON CORPORATION, Tokyo (JP)

(72) Inventor: Yu GU, Chino-shi (JP)

(21) Appl. No.: 15/091,422

(22) Filed: Apr. 5, 2016

(30) Foreign Application Priority Data
Apr. 6, 2015 (JP) 2015-078026

(51) Int. Cl. A61B 5/0245; A61B 5/00; A61B 5/04; A61B 5/0402

(52) U.S. Cl. CPC A61B 5/0245; A61B 5/0402; A61B 5/681; A61B 5/04012

(57) ABSTRACT

A biological information processing apparatus includes a pulse wave acquisition part that acquires a pulse wave signal of a user, an electrocardiogram acquisition part (heart rate acquisition part) that acquires an electrocardiogram signal of the user, and a processor (analyzer) that calculates biological information of the user based on the pulse wave signal. The processor (analyzer) analyzes the pulse wave signal based on a heart rate calculated from the electrocardiogram signal and calculates the biological information of the user if the pulse wave signal does not satisfy a predetermined condition.

### CROSS-REFERENCES TO RELATED APPLICATIONS

This application claims priority to Japanese Patent Application No. 2015-078026, filed Apr. 6, 2015, the entirety of which is herein incorporated by reference.

### BACKGROUND

1. Technical Field

The present invention relates to a biological information processing apparatus, a biological information processing system, a biological information processing method and a biological information processing program.

2. Related Art

In related art, pulsimeters attached to users for measuring pulse rates as biological information of the users are known (for example, see Patent Document 1 (JP-A-10-258040)).

The pulsimeter has a pulse wave sensor using light or ultrasonic wave and calculates a pulse rate based on changes in blood flow of a user detected by the pulse wave sensor. A pulse wave signal detected in the pulsimeter is a signal formed by superimposition of a heartbeat component signal and a body motion component signal of the user. Accordingly, when the body motion of the user is strenuous, the ratio of the body motion component signal to the heartbeat component signal is higher and it may be impossible to appropriately calculate the pulse rate.

On the other hand, the pulsimeter described in Patent Document 1 has a function of determining an SN state of the pulse wave signal, in other words, whether or not the ratio of the body motion component signal to the heartbeat component signal is higher. Accordingly, if a determination that the SN state of the pulse wave signal is good (the ratio of the body motion component signal to the heartbeat component signal is lower) is made, the calculated pulse rate is displayed, and, if a determination that the SN state is bad is made, the calculated pulse rate is not displayed.

However, in the pulsimeter described in Patent Document 1, if the determination that the SN state is bad is made, the calculated pulse rate is not displayed. For example, when the user continuously perform strenuous exercise, the ratio of the body motion component signal is higher and it is impossible to confirm the pulse rate during the exercise. In order to solve the problem, removal of the body motion component signal as noise is conceivable, however, it may be impossible to remove the body motion component signal and the pulse rate with lower reliability is calculated.

Therefore, a configuration that may calculate a pulse rate with higher reliability even when the measurement environment is bad e.g. in the bad SN state is demanded.

### SUMMARY

An advantage of some aspects of the invention is to provide a biological information processing apparatus, a biological information processing system, a biological information processing method, and a biological information processing program that may calculate a pulse rate with higher reliability.

A biological information processing apparatus according to a first aspect of the invention includes a pulse wave acquisition part that acquires a pulse wave signal of a user, an electrocardiogram acquisition part that acquires an electrocardiogram signal of the user, and a processor that calculates biological information of the user based on the pulse wave signal, wherein the processor calculates the biological information of the user based on a heart rate calculated from the electrocardiogram signal and the pulse wave signal if the pulse wave signal does not satisfy a predetermined condition.

Note that, as the biological information, a pulse rate of the user may be exemplified. Further, the electrocardiogram acquisition part acquires the electrocardiogram signal of the user.

According to the first aspect, if the pulse wave signal does not satisfy the predetermined condition, the biological information of the user is calculated based on the heart rate and the pulse wave signal of the user, and thereby, for example, compared to the case where biological information (e.g. pulse rate) is calculated only based on the pulse wave signal, the biological information (pulse rate) with higher reliability may be calculated.

In the first aspect, it is preferable that the predetermined condition includes an index of at least one of an SN ratio of the pulse wave signal and body motion of the user.

As a magnitude of the body motion of the user, exercise intensity of the user may be exemplified.

Here, if the SN ratio of the pulse wave signal is lower, the body motion noise component is larger and it is highly possible that the pulse rate calculation from the pulse rate signal is incorrect. Further, in the case where the user continuously performs exercise with higher exercise intensity, the frequency of the pulse wave signal and the frequency of the body motion signal (the above described body motion noise component) overlap and it is possible that the pulse rate calculation of the user is incorrect. On the other hand, in the first aspect, the above described predetermined condition is set based on the index of at least one of the SN ratio of the pulse wave signal and the body motion of the user, and thereby, if it is impossible to calculate the correct pulse rate, the biological information may be calculated based on the heart rate and the pulse wave signal of the user. Therefore, the biological information (pulse rate) with even higher reliability may be calculated.

In the first aspect, it is preferable that the processor derives frequency information based on the heart rate and analyzes the pulse wave signal based on the frequency information.

According to the first aspect with this configuration, the pulse wave signal is analyzed based on the frequency information of the heart rate, and thereby, the pulse wave signal of the user may be reliably analyzed.

In the first aspect, it is preferable that the frequency information is information estimated based on the heart rate and representing a frequency range in which the pulse wave signal exists.

According to the first aspect with this configuration, whether or not the pulse wave signal acquired by the pulse wave signal acquisition part exists in the range in which the pulse wave signal estimated based on the heart rate of the user exists. According to the configuration, for example, if the acquired pulse wave signal is not within the frequency range, a determination that the pulse rate of the user calculated based on the pulse wave signal of the user is incorrect may be made. Further, of the pulse wave signals existing within the range, biological information (pulse rate) based on the frequency with the highest peak may be calculated as the pulse rate of the user. Therefore, the biological information (pulse rate) with extremely high reliability may be calculated.

In the first aspect, it is preferable that a timer that times a period is provided, and the processor compares a first time when the pulse wave signal is acquired and a second time when the electrocardiogram signal is acquired, and, if a difference between the first time and the second time is within a predetermined period, analyzes the pulse wave signal based on the heart rate calculated from the electrocardiogram signal according to a comparison result and calculates the biological information.

Note that, as the above described predetermined period, 60 seconds may be exemplified.

Here, it is extremely lowly possible that the biological information (pulse rate) based on the pulse wave signal acquired after the predetermined period or more is elapsed from the second time when the electrocardiogram signal is acquired is substantially equal to the heart rate based on the electrocardiogram signal. Accordingly, even when the biological information (pulse rate) of the user is calculated based on the heart rate based on the electrocardiogram signal and the pulse wave signal after the predetermined period or more is elapsed, the calculated biological information (pulse rate) does not necessarily have high reliability.

On the other hand, according to the first aspect with the configuration described above, if the difference between the first time when the pulse wave signal is acquired and the second time when the electrocardiogram signal is acquired is within the predetermined period, the pulse wave signal is analyzed and the biological information is calculated, and thereby, the possibility of calculation of the pulse rate with extremely high reliability is higher.

In the first aspect, it is preferable that the frequency range is wider according to an elapsed time from the second time when the electrocardiogram signal is acquired.

Here, as the elapsed time from the second time when the electrocardiogram signal is acquired is longer, it is highly possible that the heart rate based on the electrocardiogram signal and the biological information (pulse rate) based on the pulse wave signal are separated from each other. Accordingly, for example, when the frequency range of the biological information (pulse wave) of the user is set to ±5×0.0625 Hz regardless of the elapsed time from the second time, a frequency peak within the range is calculated as the pulse rate though there is inherently the highest frequency peak.

On the other hand, according to the first aspect with the configuration described above, the frequency range is wider according to the elapsed time from the second time, and the highest frequency peak of the frequencies of the user may be calculated as the pulse rate. Therefore, the possibility of calculation of the pulse rate with extremely high reliability is higher.

In the first aspect, it is preferable that a report unit that prompts the user to acquire the electrocardiogram signal if the difference between the first time and the second time exceeds the predetermined period is provided.

According to the first aspect with this configuration, the user may be prompted to measure the heart rate by the report of the report unit. Therefore, the possibility of shortening of the elapsed time from the second time is higher, and the pulse rate with higher reliability may be calculated.

A biological information processing system according to a second aspect of the invention includes a detection apparatus having a pulse wave detector that detects a pulse wave signal of the user, an electrocardiogram detector that detects an electrocardiogram signal of the user, and a transmitter that transmits the pulse wave signal and the electrocardiogram signal, and the above described biological information processing apparatus, wherein the biological information processing apparatus includes a receiver that receives the pulse wave signal and the electrocardiogram signal.

According to the second aspect, the same advantages as those of the biological information processing apparatus according to the first aspect may be obtained. Further, the detection apparatus does not execute the above described processing, and thereby, the processing in the detection apparatus may be simplified.

In the second aspect, it is preferable that the detection apparatus includes a casing that houses the pulse wave detector, the electrocardiogram detector, and the transmitter, and the electrocardiogram detector includes a first surface-side electrode provided on a first surface in the casing, and a second surface-side electrode provided on a second surface different from the first surface in the casing.

According to the second aspect with this configuration, for example, when the second surface is a surface at the attachment part side in the casing, the second surface-side electrode provided on the second surface may be reliably brought into contact with the human body of the user. Further, the first surface is the opposite surface to the second surface, and the human body of the user may be easily brought into contact with the first surface-side electrode provided on the first surface with a hand or the like put thereon. Therefore, the detection and the measurement of the electrocardiogram of the user may be easily performed and the conduction path between the first surface-side electrode and the second surface-side electrode may be made longer, and the detection accuracy of the electrocardiogram may be improved.

A biological information processing method according to a third aspect of the invention includes a pulse wave acquisition step of acquiring a pulse wave signal of a user, an electrocardiogram acquisition step of acquiring an electrocardiogram signal of the user, and a processing step of calculating biological information of the user based on the pulse wave signal, wherein the processing step calculates the biological information of the user based on a heart rate calculated from the electrocardiogram signal and the pulse wave signal if the pulse wave signal does not satisfy a predetermined condition.

According to the third aspect, the same advantages as those of the biological information processing apparatus according to the first aspect and the biological information processing system according to the second aspect may be obtained.

A biological information processing program according to a fourth aspect of the invention is a biological information processing program executed by a computer and allowing the computer to function as a biological information processing apparatus including a pulse wave acquisition part that acquires a pulse wave signal of a user, an electrocardiogram acquisition part that acquires an electrocardiogram signal of the user, and a processor that calculates biological information of the user based on the pulse wave signal, wherein the processor calculates the biological information of the user based on a heart rate calculated from the electrocardiogram signal and the pulse wave signal if the pulse wave signal does not satisfy a predetermined condition.

According to the fourth aspect, the same advantages as those of the biological information processing apparatus according to the first aspect and the biological information processing system according to the second aspect may be obtained.

### BRIEF DESCRIPTION OF THE DRAWINGS

The invention will be described with reference to the accompanying drawings, wherein like numbers reference like elements.

FIG. 1 is a front view showing a biological information processing apparatus according to the first embodiment of the invention.

FIG. 2 is a rear view showing the biological information processing apparatus according to the first embodiment.

FIG. 3 is a block diagram showing a configuration of the biological information processing apparatus according to the first embodiment.

FIG. 4 is a block diagram showing a configuration of a controller of the biological information processing apparatus according to the first embodiment.

FIG. 5 is a block diagram showing a configuration of an analyzer of the controller according to the first embodiment.

FIG. 6 shows an example of an acceptable range set by a range setting part according to the first embodiment.

FIG. 7 is a flowchart showing pulsebeat specification processing according to the first embodiment.

FIG. 8 is a flowchart showing pulsebeat confirmation processing according to the first embodiment.

FIG. 9 is a flowchart showing pulsebeat fixing processing according to the first embodiment.

FIG. 10 is a flowchart showing pulsebeat specification processing according to a first modified example of the first embodiment.

FIG. 11 is a schematic diagram showing a biological information processing system according to the second embodiment of the invention.

FIG. 12 is a block diagram showing a configuration of the biological information processing system according to the second embodiment.

### DESCRIPTION OF EXEMPLARY EMBODIMENTS

### First Embodiment

As below, the first embodiment of the invention will be explained with reference to the drawings.

Schematic Configuration of Biological Information Processing Apparatus

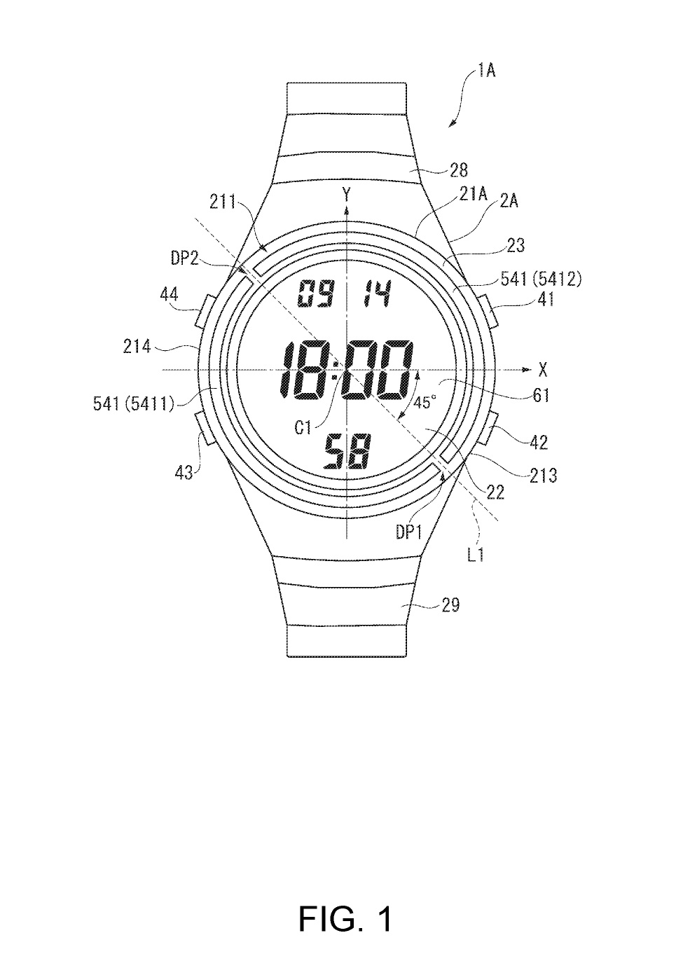

FIG. 1 is a front view showing a biological information processing apparatus 1A according to the embodiment.

The biological information processing apparatus (hereinafter, may be abbreviated as “processing apparatus”) 1A according to the embodiment is a wearable apparatus attached to an attachment part of a wrist or the like of a user and used, and detects and stores biological information of the user. Specifically, the processing apparatus 1A detects pulse wave and electrocardiogram as biological information of the user, stores the electrocardiogram and analyzes the pulse wave detected based on the electrocardiogram, and calculates and stores a pulse rate based on the analyzed pulse wave.

The processing apparatus 1A includes a casing 2A having a main body part 21A and a pair of bands 28, 29, and an apparatus main body 3 housed in the casing 2A as shown in FIG. 1.

The pair of bands 28, 29 are connected on one end and the other end of the main body part 21A in the longitudinal direction, and extend in opposite directions to each other with respect to the main body part 21A. The pair of bands 28, 29 are adapted to be fastened by a buckle (not shown) provided on an end of the band 28 (an opposite end to the connecting part to the main body part 21A). As described above, the bands 28, 29 are fastened, and thereby, the main body part 21A is attached to the attachment part. Note that the bands 28, 29 may be integrally formed with the main body part 21A. In this case, the main body part 21A corresponds to the casing 2A.

The main body part 21A houses the apparatus main body 3, which will be described later. The main body part 21A has a back surface 212 as a surface in contact with a body of the user when the processing apparatus 1A is attached to the body of the user, a front surface 211 as a surface faces the back surface 212, and a right side surface 213 and a left side surface 214 connecting the surfaces. That is, the back surface 212 is a surface on which a pulse wave sensor 531 of a pulse wave detector 53 or a light-transmissive member covering the pulse wave sensor 531, which will be described later, is provided, and the front surface 211 is a surface opposite to the back surface 212.

Of them, a display part 61 forming the apparatus main body 3 is provided nearly at the center of the front surface 211 (corresponding to a first surface), and the display part 61 is covered by a circular cover 22. Note that the front surface 211 is one surface opposite to the back surface 212 as seen along a normal of the display surface in the display part 61. Accordingly, the front surface 211 may be one flat surface or partially curved or uneven.

Further, an annular electrode placement part 23 surrounding the display part 61 and the cover 22 is provided on the front surface 211. In the electrode placement part 23, a front surface-side electrode 541 forming an electrocardiography part 54 of a measurement unit 5, which will be described later, is provided. Note that the electrode placement part 23 also functions as a bezel.

The front surface-side electrode 541 corresponds to a first surface-side electrode according to the invention and includes two electrodes 5411 and 5412. These electrodes 5411 and 5412 are respectively formed in semi-circular arc shapes and insulated from each other by an insulating material such as rubber in the electrode placement part 23. The placement of the electrodes 5411 and 5412 will be described later.

Buttons 41 to 44 of an operation unit 4 forming the apparatus main body 3 are provided on the right side surface 213 and the left side surface 214. These buttons 41 to 44 are buttons protruding and receding with respect to the main body part 21A.

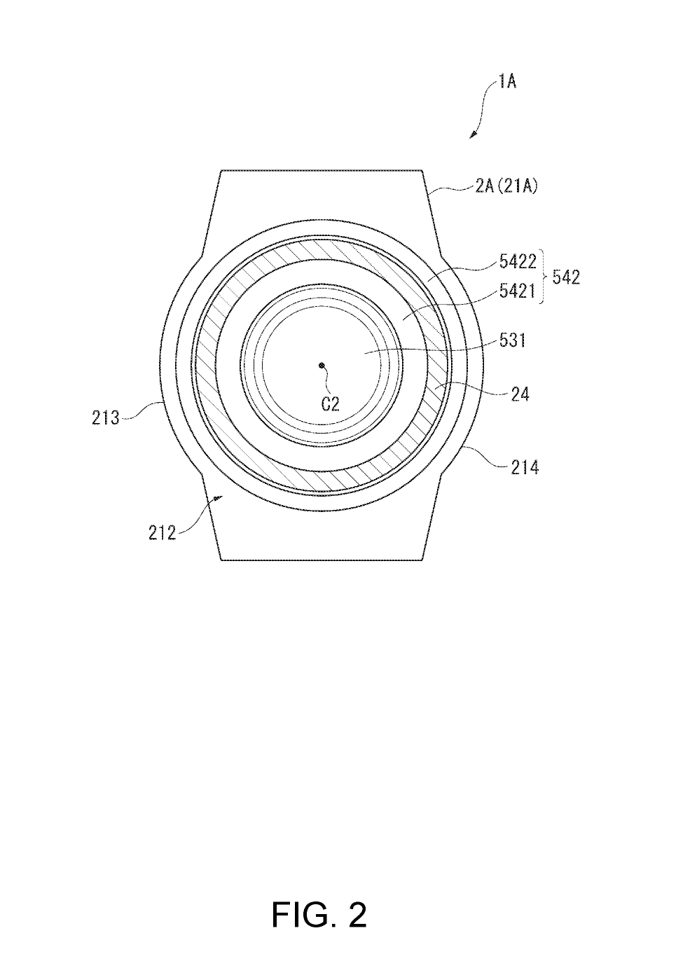

FIG. 2 is a rear view showing the processing apparatus 1A and specifically showing the back surface 212 of the main body part 21A.

The back surface 212 (corresponding to a second surface) is a surface opposed to the attachment part when the processing apparatus 1A is attached to the attachment part. In the back surface 212, the pulse wave sensor 531 and a back surface-side electrode 542 forming the electrocardiography part 54 are exposed.

The pulse wave sensor 531 is a sensor in a nearly circular shape forming the pulse wave detector 53 of the measurement unit 5, and provided nearly at the center of the back surface 212. Note that the pulse wave sensor 531 may be directly provided on the back surface 212, and the light-transmissive member provided on the apparatus main body 3 provided within the main body part 21A and covering a light emitting device and a light receiving device of the pulse wave sensor 531 may be attached to the back surface 212.

The back surface-side electrode 542 corresponds to a second surface-side electrode according to the invention and includes two electrodes 5421, 5422. Of them, the electrode 5421 is formed in a nearly circular shape and provided to be exposed in a position surrounding the pulse wave sensor 531. Further, the electrode 5422 is formed in a nearly circular shape and provided to be exposed in a position surrounding the electrode 5421 via an insulator 24.

That is, the electrodes 5421, 5422 are coaxially provided with a center C2 of the circular pulse wave sensor 531 at the center.

### Configuration of Apparatus Main Body

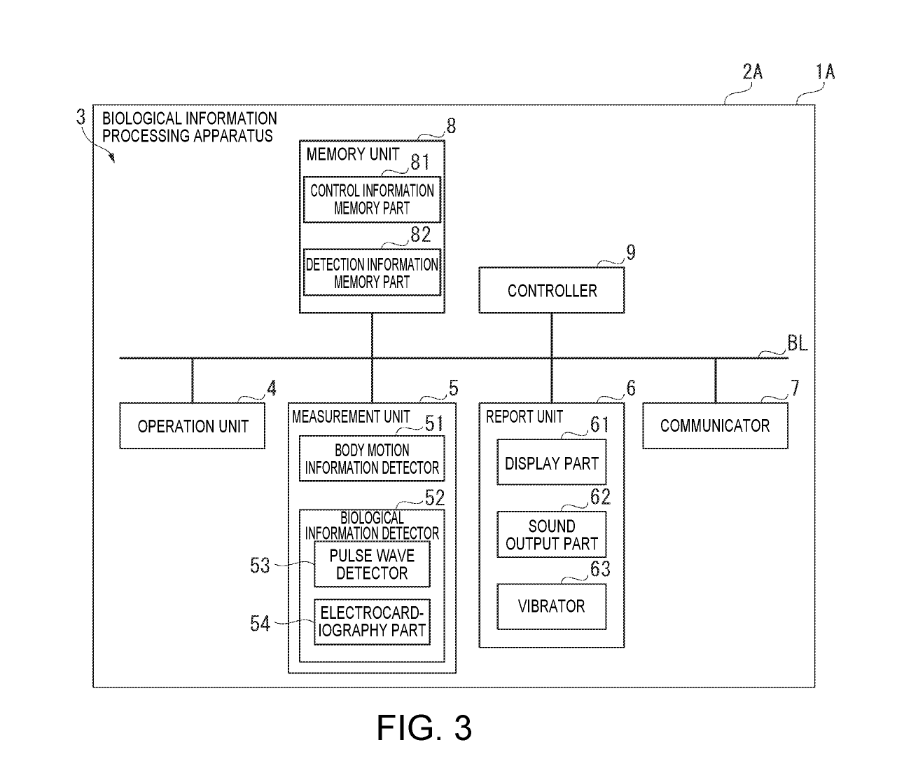

FIG. 3 is a block diagram showing a configuration of the processing apparatus 1A.

As shown in FIG. 3, the apparatus main body 3 includes the operation unit 4, the measurement unit 5, a report unit 6, a communicator 7, a memory unit 8, and the controller 9.

### Configuration of Operation Unit

The operation unit 4 includes the buttons 41 to 44, and outputs operation signals in response to input operations for the buttons 41 to 44 to the controller 9. Note that the operation unit 4 may have a configuration with a touch panel provided on the display part 61 of the report unit 6, which will be described later, or a configuration that detects tap operation of the user, not the configuration with the buttons.

### Configuration of Measurement Unit

The measurement unit 5 includes a body motion information detector 51 and a biological information detector that respectively operate under the control of the controller 9.

The body motion information detector 51 detects body motion information representing body motion of the user and outputs the body motion information to the controller 9. In the embodiment, the body motion information detector 51 detects an acceleration signal changing with the body motion of the user as body motion information. Note that the body motion information detector 51 may detect an angular velocity changing with the body motion of the user in addition to the acceleration.

The biological information detector 52 includes the pulse wave detector 53 and the electrocardiography part 54.

### Configuration of Pulse Wave Detector

The pulse wave detector 53 includes the pulse wave sensor 531 and detects pulse wave of the user under the control of the controller 9. The pulse wave sensor 531 is a photoelectric sensor having the light emitting device such as an LED (Light Emitting Diode), the light receiving device such as a photodiode, and the light-transmissive member covering the devices. In the pulse wave sensor, light radiated toward a living organism by the light emitting device is received by the light receiving device via a vessel of the living organism. A signal showing a change over time in an amount of light received by the light receiving device is output as the pulse wave signal to the controller 9, which will be described later, and the controller 9 analyzes the pulse wave signal, and thereby, the pulse rate is calculated.

### Configuration of Electrocardiography Part

The electrocardiography part 54 corresponds to an electrocardiogram detector according to the invention, and detects electrocardiogram of the user and outputs an electrocardiogram signal showing the electrocardiogram to the controller 9. The electrocardiography part 54 includes the front surface-side electrode 541 and the back surface-side electrode 542. Operation amplifiers (not shown) are respectively connected to the front surface-side electrode 541 and the back surface-side electrode 542, and amplify signals input to the respective electrodes 541, 542. Then, the electrocardiography part 54 processes the amplified signals and outputs an electrocardiogram signal based on the signals to the controller 9. Specifically, the electrocardiography part 54 filters the input signal and removes noise components and outputs the obtained electrocardiogram signal to the controller 9.

### Configuration of Report Unit

The report unit 6 reports various kinds of information to the user under the control of the controller 9. The report unit 6 includes the display part 61, a sound output part 62, and a vibrator 63.

The display part 61 includes various display panels of liquid crystal or the like and displays information input from the controller 9. For example, the display part 61 displays the body motion information and the biological information (pulse rate) detected and analyzed by the measurement unit 5. Further, the display part 61 displays presentation information generated by the controller 9.

The sound output part 62 includes sound output means such as a speaker and outputs sound in response to a sound signal input from the controller 9.

The vibrator 63 includes a motor operatively controlled by the controller 9, and reports e.g. a warning to the user by vibration generated by driving of the motor.

### Configuration of Communicator

The communicator 7 includes a communication module that can communicate with an external apparatus. The communicator 7 transmits the respectively detected and measured body motion information and biological information to the external apparatus on regular basis, and outputs information received from the external apparatus to the controller 9. Note that, in the embodiment, the communicator 7 wirelessly communicates with the external apparatus by a near field communication system, however, may communicate with the external apparatus via a relay unit such as a cradle and a cable. Further, the communicator 7 may communicate with the external apparatus via a network.

### Configuration of Memory Unit

The memory unit 8 includes memory means such as a flash memory and includes a control information memory part 81 and a detection information memory part 82.

The control information memory part 81 stores control information including various programs and data necessary for the operation of the processing apparatus 1A. As the programs, a control program for controlling the processing apparatus 1A and a biological information processing program for execution of pulsebeat specification processing, pulsebeat confirmation processing, and pulsebeat fixing processing, which will be described later, are stored.

The detection information memory part 82 stores the body motion information and the biological information detected by the measurement unit 5 and analysis results (e.g. pulse rate and heart rate) of the body motion information and the biological information by the controller 9. The detection information memory part 82 sequentially stores the information and, if its memory capacity is insufficient, overwrites the earliest stored information with newly acquired information.

### Configuration of Controller

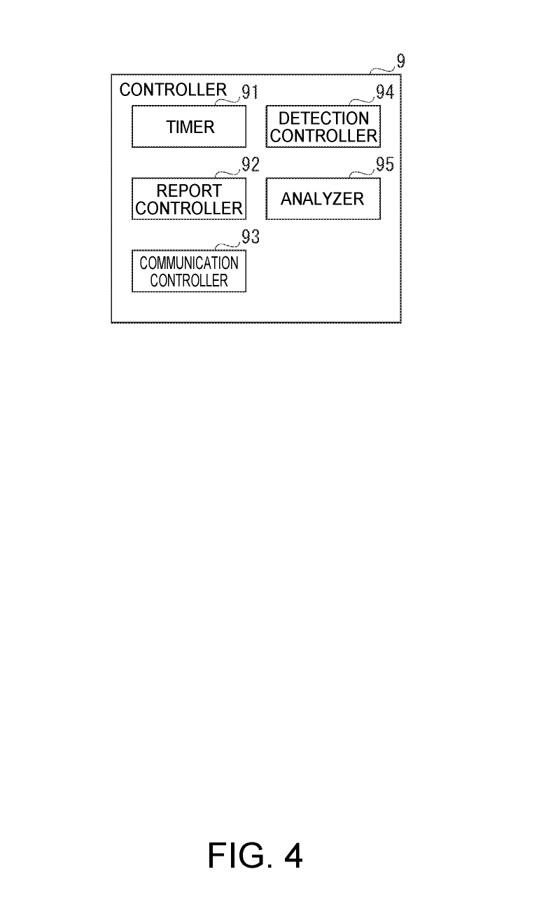

FIG. 4 is a block diagram showing a configuration of the controller 9.

The controller 9 includes a processing circuit and controls the operation of the processing apparatus 1A autonomously or in response to the operation signal input from the operation unit 4. The controller 9 controls e.g. the measurement unit 5 to detect the body motion information and the biological information.

As shown in FIG. 4, the controller 9 includes a timer 91, a report controller 92, a communication controller 93, a detection controller 94, and an analyzer 95 as functional parts realized by the processing circuit executing the programs stored in the control information memory part 81.

### Configuration of Timer, Report Controller, and Communication Controller

The timer 91 times current date and time. Further, the timer times an elapsed time after the SN ratio of the pulse wave acquired by a pulse wave acquisition part 981 falls below a predetermined value.

The report controller 92 controls the operation of the report unit 6. For example, the report controller 92 allows the report unit 6 to report an operation status of the processing apparatus 1A and presentation information containing display and sound representing detection results by the measurement unit 5 etc. Further, the report controller 92 drives a motor of the vibrator 63 as appropriate and allows the unit to report predetermined information by the vibration generated by the driving of the motor.

The communication controller 93 controls the operation of the communicator 7.

### Configuration of Detection Controller

The detection controller 94 controls the operation of the measurement unit 5. For example, the detection controller 94 allows the body motion information detector 51 to detect the body motion of the user and allows the pulse wave detector 53 to detect the pulse wave of the user. Then, the detection controller 94 allows the detection information memory part 82 to store the acceleration signal showing the body motion and the pulse wave signal showing the pulse wave with the current date and time.

Further, the detection controller 94 allows the electrocardiography part 54 to measure the electrocardiogram and allows the detection information memory part 82 to store the electrocardiogram signal showing the measured electrocardiogram with the current date and time. Note that the detection controller 94 may allow the detection information memory part 82 to store the pulse rate calculated based on the pulse wave signal as the biological information with the current date and time.

### Configuration of Analyzer

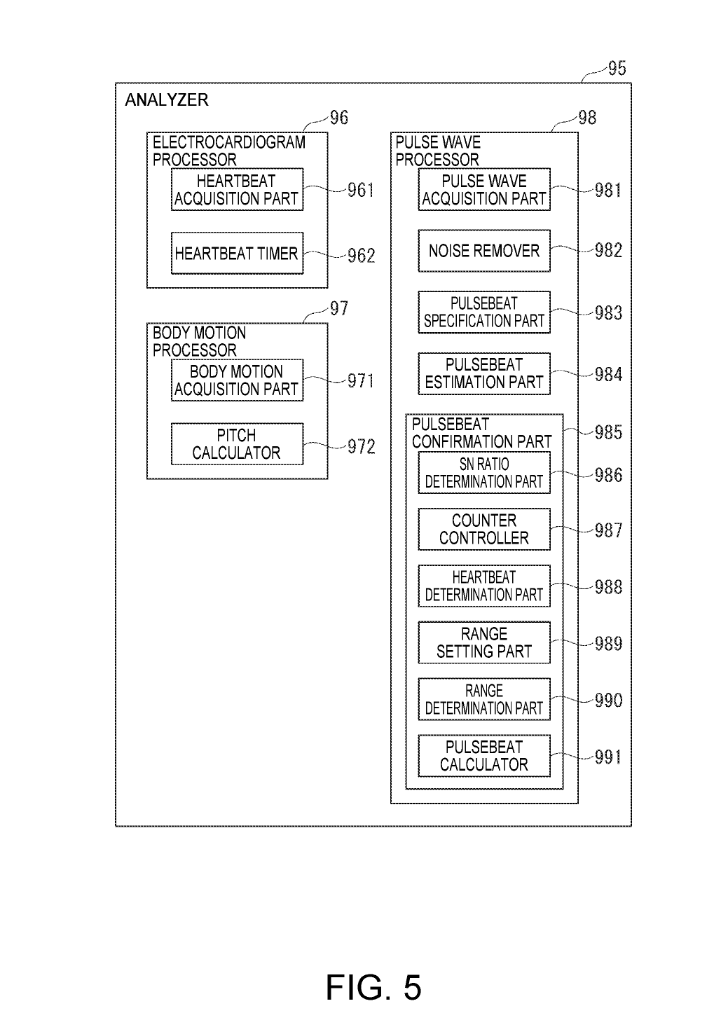

FIG. 5 is a block diagram showing a configuration of the analyzer 95.

The analyzer 95 analyzes the body motion information and the biological information input from the body motion information detector 51 and the biological information detector 52. Specifically, the analyzer 95 calculates the pulse rate of the user based on the pulse wave signal input from the pulse wave detector 53 and the acceleration signal input from the body motion information detector 51. For example, the analyzer 95 removes the body motion noise component based on the acceleration signal from the pulse wave signal and obtains the heartbeat signal. Then, the analyzer performs a frequency analysis of FFT (Fast Fourier Transform) on the heartbeat signal, extracts the frequency of the pulsebeat from the obtained analysis result (power spectrum), and calculates the pulse rate based on the frequency of the pulsebeat. Note that the analyzer 95 may calculate the pulse rate by another method, not limited to the calculation of the pulse rate.

The analyzer 95 includes an electrocardiogram processor 96, a body motion processor 97, and a pulse wave processor 98, and executes the pulsebeat confirmation processing for confirmation of the pulse rate calculated by the above described method and the pulsebeat fixing processing for fixing the pulse rate.

### Configuration of Electrocardiogram Processor

The electrocardiogram processor 96 includes a heartbeat acquisition part 961 and a heartbeat timer 962.

The heartbeat acquisition part 961 receives the electrocardiogram signal transmitted from the electrocardiography part 54 and acquires the heart rate of the user based on the electrocardiogram signal. Further, the heartbeat acquisition part 961 allows the detection information memory part 82 to store the heart rate with the time when the electrocardiogram is acquired.

The heartbeat timer 962 times an elapsed time after the heart rate of the user is acquired by the heartbeat acquisition part 961.

### Configuration of Body Motion Processor

The body motion processor 97 includes a body motion acquisition part 971 and a pitch calculator 972.

The body motion acquisition part 971 receives the acceleration signal transmitted from the body motion information detector 51 and acquires the body motion information of the user based on the acceleration signal.

The pitch calculator 972 calculates the pace (pitch) of the user based on the acceleration signal. For example, the pitch calculator 972 performs the frequency analysis on the acceleration signal, extracts the frequency of the body motion from the obtained analysis result, and calculates the pace based on the frequency of the body motion.

### Configuration of Pulse Wave Processor

The pulse wave processor 98 includes a function of acquiring the pulse wave signal detected by the pulse wave detector 53 and calculating the pulse rate of the user based on the pulse wave signal and the acceleration signal. The pulse wave processor 98 includes the pulse wave acquisition part 981, a noise remover 982, a pulsebeat specification part 983, a pulsebeat estimation part 984, and a pulsebeat confirmation part 985.

### Configurations of Pulse Wave Acquisition Part and Noise Remover

The pulse wave acquisition part 981 acquires the pulse wave signal detected by the pulse wave detector 53. Specifically, the pulse wave acquisition part 981 amplifies and AD-converts the pulse wave signal and shapes the AD-converted pulse wave signal.

The noise remover 982 amplifies and AD-converts the acceleration signal acquired by the body motion acquisition part 971 and shapes the AD-converted acceleration signal. Then, the remover removes the shaped acceleration signal as the body motion noise component from the pulse wave signal shaped by the pulse wave acquisition part 981.

### Configuration of Pulsebeat Specification Part

The pulsebeat specification part 983 specifies the pulse rate of the user based on the pulse wave signal after removal of the body motion noise component by the noise remover 982 (hereinafter, may be referred to as “heartbeat signal”) and executes an determination as to whether or not the pulse rate has been successfully specified. Specifically, if the body motion noise component has been completely removed, the pulsebeat specification part 983 performs the frequency analysis of FFT or the like on the heartbeat signal, extracts the frequency of the pulsebeat from the obtained analysis result, calculates the pulse rate based on the frequency of the pulsebeat, and specifies the calculated pulse rate. That is, if the body motion noise component has been completely removed by the noise remover 982 (the above described predetermined condition is satisfied), the pulsebeat specification part 983 may fix the pulse rate and determines that the pulse rate has been successfully specified.

### Configuration of Pulsebeat Estimation Part

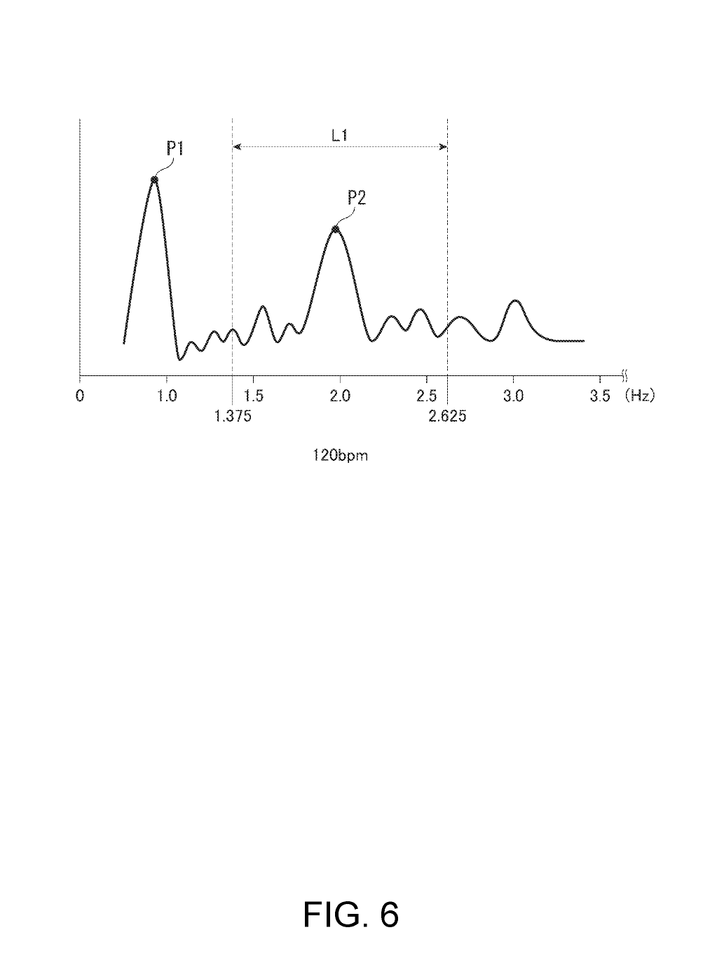

On the other hand, if the body motion noise component has not been completely removed by the noise remover 982, in other words, if a determination that the pulse rate has not successfully been specified is made by the pulsebeat specification part 983, the pulsebeat estimation part 984 estimates the pulse rate of the user. Specifically, the pulsebeat estimation part 984 estimates the frequency P1 at the maximum amplitude (see FIG. 6) of the heartbeat signals from which the body motion noise components have not been removed as the current pulse rate of the user.

### Configuration of Pulsebeat Confirmation Part

The pulsebeat confirmation part 985 determines whether or not the pulse rate estimated by the pulsebeat estimation part 984 is correct based on a pulsebeat confirmation program stored in the control information memory part 81, and, if the possibility of incorrectness is higher, analyzes the pulse wave signal (heartbeat signal) of the user based on the heart rate calculated by the electrocardiogram processor 96 and calculates the pulse rate as the biological information of the user. The pulsebeat confirmation part 985 corresponds to a processor according to the invention, and includes an SN ratio determination part 986, a counter controller 987, a heartbeat determination part 988, a range setting part 989, a range determination part 990, and a pulsebeat calculator 991.

Configuration of SN ratio Determination Part

The SN ratio determination part 986 detects the SN ratio of the pulse wave signal of the user acquired by the pulse wave acquisition part 981 and determines whether or not the SN ratio is larger than a predetermined threshold value.

Here, the SN ratio is a ratio of an amount of signal to an amount of noise, and a larger SN ratio refers to a smaller influence of noise and a smaller SN ratio refers to a larger influence of noise. That is, in the embodiment, the SN ratio is a ratio of the pulse wave signal to the body motion noise signal. Accordingly, an SN ratio larger than the predetermined threshold value refers to a less body motion noise component in the pulse wave signal.

### Configuration of Counter Controller

The counter controller 987 resets the counter value if the SN ratio determination part 986 determines that the SN ratio is larger than the predetermined threshold value, and increments the counter value if the part determines that the SN ratio is smaller than the predetermined threshold value. Further, the counter controller 987 executes a determination as to whether or not the counter value exceeds a predetermined value.

Note that the counter value is an elapsed time after the determination that the SN ratio is determined to be smaller than the predetermined threshold value is made by the SN ratio determination part 986. The counter value is set to e.g. five seconds.

### Configuration of Heartbeat Determination Part

If a determination that the counter value exceeds the predetermined value by the counter controller 987 is made, the heartbeat determination part 988 determines whether or not the heart rate most recently measured is stored in the detection information memory part 82. Specifically, the heartbeat determination part 988 compares the time when the pulse wave acquisition part 981 acquires the pulse wave signal (first time) and the time when the signal is stored with the heart rate of the user in the detection information memory part 82 (second time), and determines whether or not the difference between the first time and the second time is within a predetermined period (e.g. within 60 seconds). In other words, the heartbeat determination part 988 determines whether or not there is a heart rate stored in the detection information memory part 82 within 60 seconds from the first time when the pulse wave signal is acquired.

Note that, if a determination that there is no heart rate measured within the predetermined period is made by the determination of the heartbeat determination part 988, the report controller 92 allows the display part 61 to display a screen prompting the user to measure the heart rate.

### Configuration of Range Setting Part

If a determination that there is a heart rate measured within the predetermined period is made by the determination of the heartbeat determination part 988, the range setting part 989 sets an acceptable frequency range in which the pulse wave signal according to the elapsed time after the electrocardiogram measurement exists.

Here, the acceptable frequency range is explained. The acceptable frequency range is set to a range including the frequency of the heart rate stored in the detection information memory part 82 and the neighborhood frequencies. Specifically, within one second from the time when the electrocardiogram of the heart rate is measured, the range setting part 989 does not set the range, but sets the heart rate based on the electrocardiogram as the pulse rate. Further, if the elapsed time from the time of electrocardiogram measurement is equal to or more than two seconds and less than five seconds, the range setting part 989 sets the above described range to “the frequency of the heartbeat and the frequency of the heart rate ±5×0.0625 Hz”. Furthermore, if the elapsed time from the time of electrocardiogram measurement is equal to or more than five seconds and less than 10 seconds, the range setting part 989 sets the above described range to “the frequency of the heartbeat and the frequency of the heart rate ±10×0.0625 Hz”. In addition, if the elapsed time from the time of electrocardiogram measurement is equal to or more than 10 seconds and less than 20 seconds, the range setting part 989 sets the above described range to “the frequency of the heartbeat and the frequency of the heart rate ±16×0.0625 Hz”. Or, if the elapsed time from the time of electrocardiogram measurement is equal to or more than 20 seconds and less than 60 seconds, the range setting part 989 sets the above described range to “the frequency of the heartbeat and the frequency of the heart rate ±20×0.0625 Hz”.

Note that, if 60 seconds or more elapse, the determination that there is no heart rate is made by the heartbeat determination part 988, and the screen prompting the user to measure the heart rate is displayed on the display part 61 as described above.

FIG. 6 shows an example of the acceptable range set by the range setting part 989. Note that a value obtained by multiplication of the numeric value of the pulse wave (Hz) by “60” is the heart rate.

For example, when the heart rate stored in the detection information memory part 82 is “120 bpm” and the above described elapsed time is “seven seconds”, as shown in FIG. 6, an acceptable range L1 is set to “the frequency of the heartbeat (2.0 Hz) and the frequency of the heart rate ±10×0.0625 Hz”. Specifically, the range setting part 989 sets the acceptable range L1 equal to or more than 1.375 Hz and less than 2.625 Hz. That is, the frequency range of the acceptable range L1 set by the range setting part 989 corresponds to frequency information according to the invention.

### Configuration of Range Determination Part

Returning to FIG. 5, the range determination part 990 determines whether or not the pulse wave estimated in the pulsebeat estimation part 984 is within the acceptable range set by the range setting part 989. For example, the range determination part 990 determines whether or not the frequency P1 of the pulse wave according to the pulse rate estimated in the pulsebeat estimation part 984 is within the acceptable range L1. Thereby, as shown in FIG. 6, if a determination that the frequency P1 is not within the acceptable range L1 is made by the range determination part 990, processing by the pulsebeat confirmation part 985, which will be described later, is executed.

On the other hand, if a determination that the frequency P1 is within the acceptable range L1 is made by the range determination part 990, the pulse rate estimated in the pulsebeat estimation part 984 is fixed as the pulse rate of the user, and the pulsebeat fixing processing and the pulsebeat confirmation processing are ended.

### Configuration of Pulsebeat Calculator

If the determination that the pulse wave frequency P1 is not within the acceptable range L1 is made by the range determination part 990, the pulsebeat calculator 991 does not employ the pulse rate estimated by the pulsebeat estimation part 984, but newly calculates a pulse rate of the user. Specifically, the pulsebeat calculator 991 detects a frequency corresponding to the peak value within the acceptable range in the pulse wave signal spectrum. In other words, the pulsebeat calculator 991 detects a frequency P2 with the highest peak within the acceptable range L1 shown in FIG. 6. Then, the pulsebeat calculator 991 calculates the pulse rate based on the frequency P2. For example, the calculator calculates a value by multiplication of “2 Hz” as the value of the frequency P2 by “60” as the pulse rate.

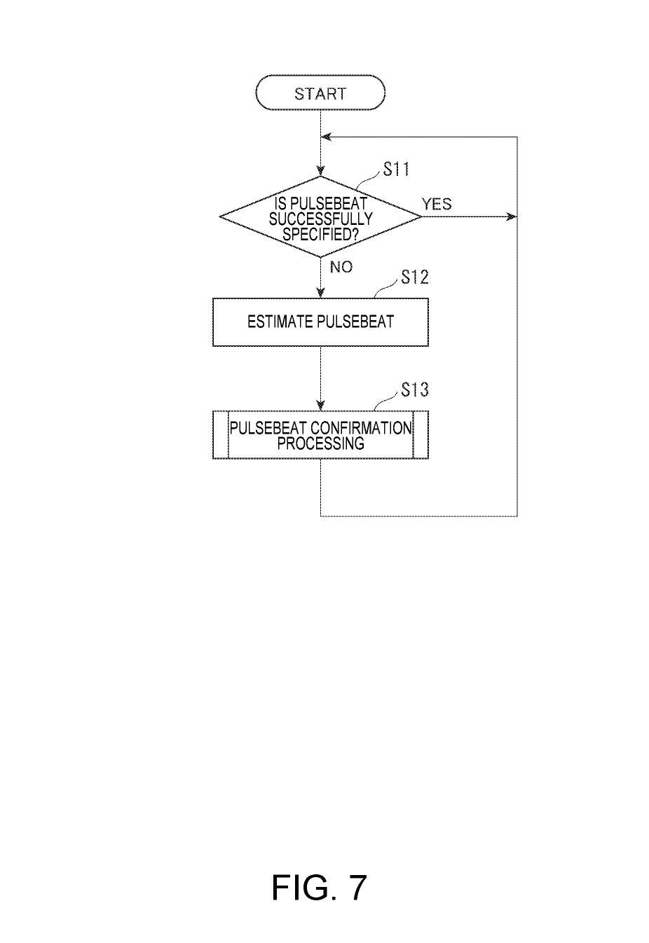

FIG. 7 is a flowchart showing a processing procedure of pulsebeat confirmation processing executed by the pulse wave processor 98 of the analyzer 95.

The pulse wave processor 98 executes the following pulsebeat confirmation processing based on a program stored in the memory unit 8. The pulsebeat confirmation processing is processing of analyzing the pulse wave signal based on the heart rate calculated from the electrocardiogram signal and calculating the pulse rate of the user if the pulse wave signal does not satisfy a predetermined condition. In other words, the pulsebeat confirmation processing is processing of determining whether or not the pulse rate estimated by the pulse wave processor 98 is a value with high reliability, and, if the value is not the value with high reliability, calculating a pulse rate with high reliability by executing various kinds of processing and fixing the pulse rate as the pulse rate of the user.

Specifically, as shown in FIG. 7, the pulsebeat specification part 983 in the pulse wave processor 98 specifies the pulse rate of the user based on the pulsebeat signal after removal of the body motion noise component by the noise remover 982 and executes a determination as to whether or not the pulse rate has been successfully specified (step S11).

In the determination processing at step S11, if a determination that the pulse rate has been successfully specified is made, the pulse wave processor 98 returns the processing to step S11. Note that, in the determination processing at step S11, if the determination that the pulse rate has been successfully specified is made, the specified pulse rate is displayed on the display part 61 under the control of the report controller 92.

On the other hand, in the determination processing at step S11, if a determination that the pulse rate has not successfully been specified is made, the pulsebeat estimation part 984 estimates the pulse rate of the user (step S12). Specifically, the pulsebeat estimation part 984 estimates the frequency P1 having the maximum amplitude (see FIG. 6) of the pulsebeat signals before removable of the body motion noise component as the current pulse rate of the user.

Then, the pulse wave processor 98 executes the pulsebeat confirmation processing (step S13). Note that the estimation processing of the pulse rate at step S12 is not an essential configuration, and may be omitted. For example, on the basis of the SN ratio of the pulse wave signal from the pulse wave detector 53, if the value of the SN ratio is equal to or more than a threshold value, the pulse rate may be calculated based on the pulse wave signal from the pulse wave detector 53 and, if the value is smaller than the threshold value, the processing at step S13 may be executed for specification of the pulse rate.

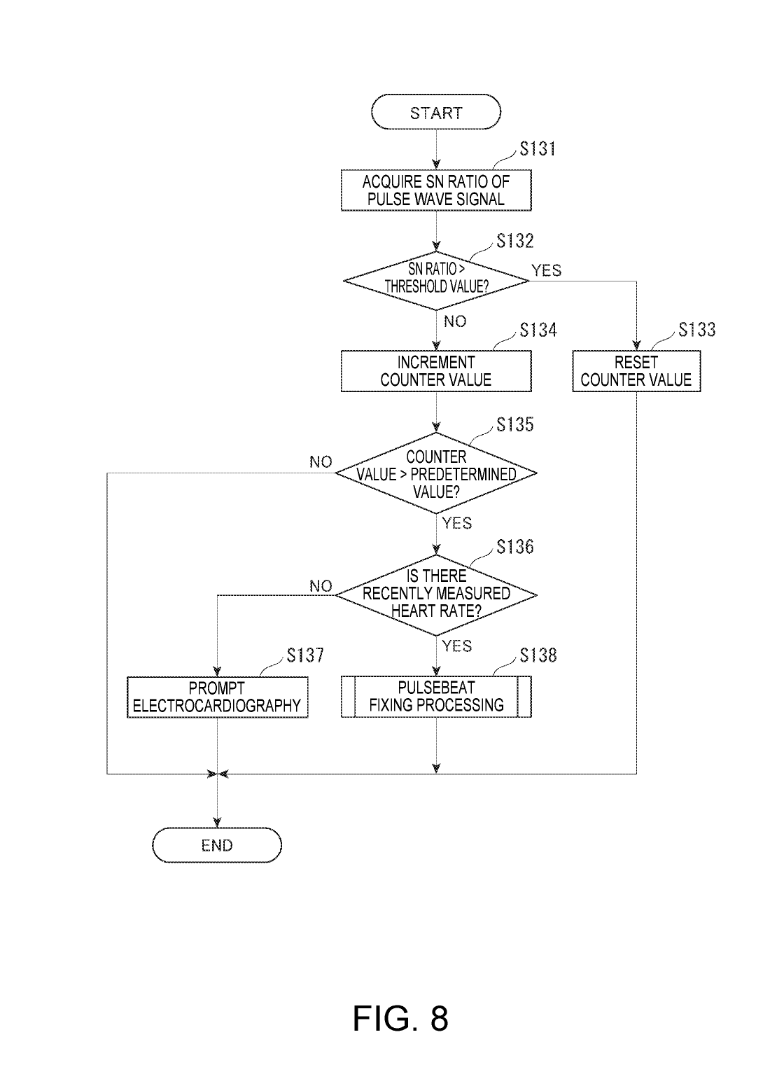

FIG. 8 is a flowchart showing the processing procedure of the pulsebeat confirmation processing.

The pulsebeat confirmation processing executed at step S13 will be described in detail.

First, the SN ratio determination part 986 detects the SN ratio of the pulse wave signal of the user acquired by the pulse wave acquisition part 981 (step S131) and determines whether or not the SN ratio is larger than the predetermined threshold value (step S132).

In the determination processing at step S132, if a determination that the SN ratio is larger than the predetermined threshold value is made, the counter controller 987 resets the counter value (step S133). Then, the pulsebeat confirmation processing is ended and the pulse rate estimated in the pulsebeat estimation part 984 is fixed as the pulse rate of the user.

On the other hand, in the determination processing at step S132, if a determination that the SN ratio is not larger than the predetermined threshold value is made, the counter controller 987 increments the counter value (step S134).

Then, the counter controller 987 determines whether or not the counter value exceeds a predetermined value (step S135).

In the determination processing at step S135, if a determination that the counter value does not exceed the predetermined value is made, the pulsebeat confirmation processing is ended.

On the other hand, in the determination processing at step S135, if a determination that the counter value exceeds the predetermined value is made, the heartbeat determination part 988 determines whether or not the heart rate most recently measured is stored in the detection information memory part (step S136).

In the determination processing at step S136, if a determination that the heart rate most recently measured is not stored in the detection information memory part 82 is made, the report controller 92 allows the display part 61 to display a screen prompting the user to measure the heart rate (step S137).

On the other hand, in the determination processing at step S136, if a determination that the heart rate most recently measured is stored in the detection information memory part 82 is made, the pulsebeat confirmation part 985 executes the pulsebeat confirmation processing (step S138).

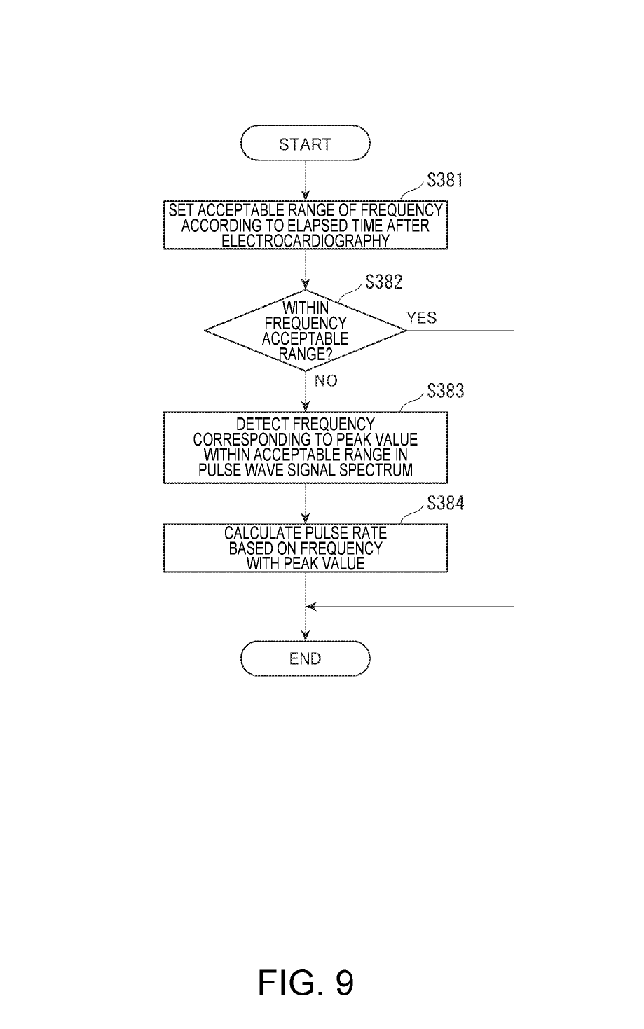

FIG. 9 is a flowchart showing a processing procedure of the pulsebeat fixing processing.

The pulsebeat fixing processing executed at step S138 will be described later in detail.

First, the range setting part 989 sets an acceptable frequency range in which the pulse wave signal according to the elapsed time after the electrocardiogram measurement exists (step S381).

Then, the range determination part 990 determines whether or not the pulse wave estimated in the pulsebeat estimation part 984 is within the acceptable range set by the range setting part 989 (step S382).

In the determination processing at step S382, if a determination that the pulse wave is within the acceptable range is made, the pulse rate estimated by the pulsebeat estimation part 984 is fixed as the pulse rate of the user and the pulsebeat fixing processing and the pulsebeat confirmation processing are ended.

On the other hand, in the determination processing at step S382, if a determination that the pulse wave is not within the acceptable range is made, the pulsebeat calculator 991 detects frequencies corresponding to the peak values within the acceptable range in the pulse wave signal spectrum (step S383).

Then, the pulsebeat calculator 991 calculates the pulse rate based on the frequency corresponding to the value with the highest peak within the acceptable range (step S384), and the pulsebeat fixing processing is ended.

### Advantages of First Embodiment

According to the above described biological information processing apparatus 1A, the following advantages are obtained.

If the pulse wave signal does not satisfy the predetermined condition, in other words, if the SN ratio of the pulse wave signal is lower, the pulse rate of the user is calculated based on the heart rate and the pulse wave signal of the user, and thereby, the pulse rate with higher reliability may be calculated compared to e.g. the case where the pulse rate is calculated only based on the pulse wave signal.

Here, if the SN ratio of the pulse wave signal is lower, the body motion noise component is stronger and it is highly possible that the pulse rate calculation from the pulse wave signal is incorrect. If the frequency of the pulse wave signal and the frequency of the body motion signal (the above described body motion noise component) overlap, it is possible that the pulse wave calculation of the user is incorrect. On the other hand, in the embodiment, the predetermined condition is set using the SN ratio of the pulse wave signal as an index, and, if it is impossible to calculate the correct pulse rate, the pulse rate may be calculated based on the heart rate and the pulse wave signal of the user. Therefore, the pulse rate with even higher reliability may be calculated.

Further, the pulse wave signal is analyzed based on the frequency information of the heart rate, and thereby, the pulse wave signal of the user may be reliably analyzed.

Specifically, the range determination part 990 may determine whether or not the pulse wave signal acquired by the pulse wave acquisition part 981 exists in the frequency range in which the pulse wave signal estimated based on the heart rate of the user exists (within the acceptable range L1). According to the configuration, for example, if the acquired pulse wave signal is not within the frequency range, a determination that the pulse rate of the user calculated based on the pulse wave signal of the user is incorrect may be made. Further, of the pulse wave signals existing within the range, the pulse rate based on the frequency P2 with the highest peak may be calculated as the pulse rate of the user. Therefore, the pulse rate with extremely high reliability may be calculated.

It is lowly possible that the pulse rate based on the pulse wave signal acquired after a predetermined period (60 seconds) or more elapses from the second time when the electrocardiogram signal is acquired is substantially equal to the heart rate based on the electrocardiogram signal. Accordingly, if the pulse rate of the user is calculated based on the heart rate based on the electrocardiogram signal and the pulse wave signal after 60 seconds or more elapse, the calculated pulse rate does not necessarily have high reliability.

On the other hand, according to the embodiment, if the difference between the first time when the pulse wave signal is acquired and the second time when the electrocardiogram signal is acquired is within the predetermined period (60 seconds), the pulse wave signal is analyzed and the pulse rate is calculated, and thereby, the possibility of calculation of the pulse rate with extremely high reliability is higher.

As the elapsed time from the second time when the electrocardiogram signal is acquired is longer, it is highly possible that the heart rate based on the electrocardiogram signal and the pulse rate based on the pulse wave signal are separated from each other. Accordingly, for example, when the frequency range of the pulse wave of the user is set to ±5×0.0625 Hz regardless of the elapsed time from the second time, a frequency peak within the range is calculated as the pulse rate though there is inherently the highest frequency peak.

On the other hand, according to the embodiment, the frequency range (acceptable range L1) is set to be wider according to the elapsed time from the second time, and the highest peak of the frequency P2 of the frequencies of the user may be calculated as the pulse rate. Therefore, the possibility of calculation of the pulse rate with extremely high reliability is higher.

Under the control of the report controller 92, the screen prompting the user to measure the heart rate is displayed on the display part 61, and thereby, the user may be prompted to measure the heart rate. Therefore, the possibility of shortening of the elapsed time from the second time is higher, and the pulse rate with higher reliability may be calculated.

The back surface 212 is the surface at the attachment part side in the casing 2A, and the back surface-side electrode 542 provided on the back surface 212 may be reliably brought into contact with the human body of the user. Further, the front surface 211 is the opposite surface to the back surface 212, and the human body of the user may be easily brought into contact with the front surface-side electrode 541 provided on the front surface 211 with a hand or the like put thereon. Therefore, the detection and the measurement of the electrocardiogram of the user may be easily performed and the conduction path between the front surface-side electrode 541 and the back surface-side electrode 542 may be made longer, and the detection accuracy of the electrocardiogram may be improved.

### First Modification of First Embodiment

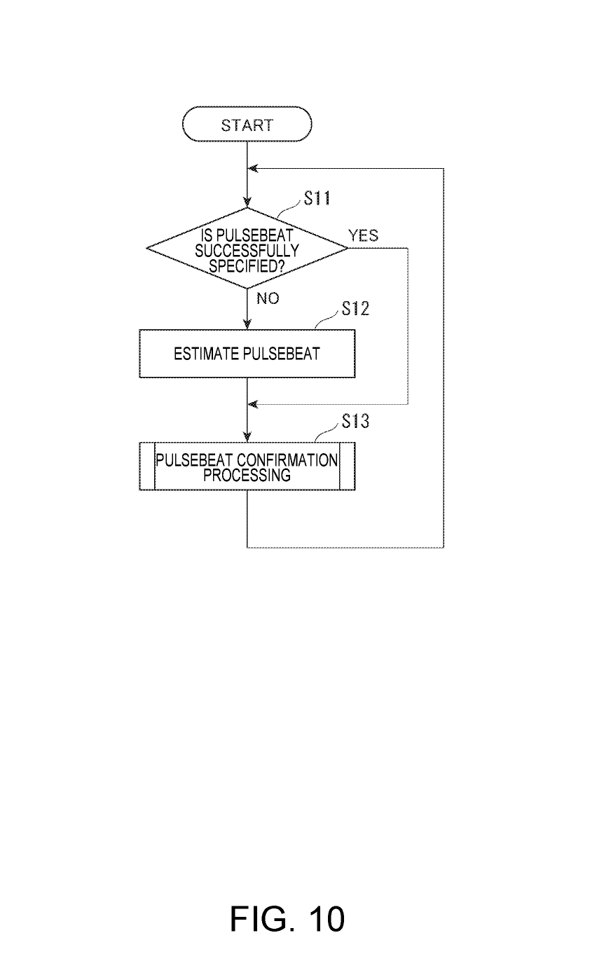

FIG. 10 is a flowchart showing a processing procedure of pulsebeat confirmation processing executed by the pulse wave processor 98 of the analyzer 95.

In the first embodiment, if the determination that the pulse rate has been successfully specified is made in the determination processing at step S11, the pulse wave processor 98 returns the processing to step S11, and the specified pulse rate is displayed on the display part 61 under the control of the report controller 92. However, the processing is not limited to that. For example, as shown in FIG. 10, even in the case where the determination that the pulse rate has been successfully specified is made in the determination processing at step S11, the pulsebeat confirmation processing (step S13) may be executed.

According to the configuration, in either case where the pulse rate has been successfully specified or the pulse rate is estimated, the pulsebeat confirmation processing may be executed. Therefore, compared to the above described first embodiment, the more correct pulse rate may be calculated and presented to the user.

### Second Modification of First Embodiment

In the above described first embodiment, the range setting part 989 sets an acceptable frequency range in which the pulse wave signal according to the elapsed time after the electrocardiogram measurement exists at step S381, and the range determination part 990 determines whether or not the pulse wave estimated in the pulsebeat estimation part 984 is within the acceptable range set by the range setting part 989 at step S382. However, the processing is not limited to that. For example, the range setting part 989 may set an acceptable range of pulse rate according to the elapsed time after the electrocardiogram measurement. Further, the range determination part 990 may determine whether or not the pulse rate is within the acceptable range of pulse rate. Also, in this case, the same advantages as those of the above described first embodiment and first modification of the first embodiment may be obtained.

### Second Embodiment

Next, the second embodiment of the invention will be explained.

A biological information processing system according to the embodiment executes the biological information processing executed by the biological information processing apparatus 1A using a detection apparatus and an information processing apparatus. The detection apparatus has substantially the same configuration as that of the biological information processing apparatus 1A. Further, the information processing apparatus has substantially the same function as that of the controller 9 of the biological information processing apparatus 1A. In this regard, the biological information processing apparatus 1A is different from the biological information processing system.

Note that, in the following explanation, the same or substantially the same parts as those described above have the same signs and their explanation will be omitted.

### Configuration of Biological Information Processing System

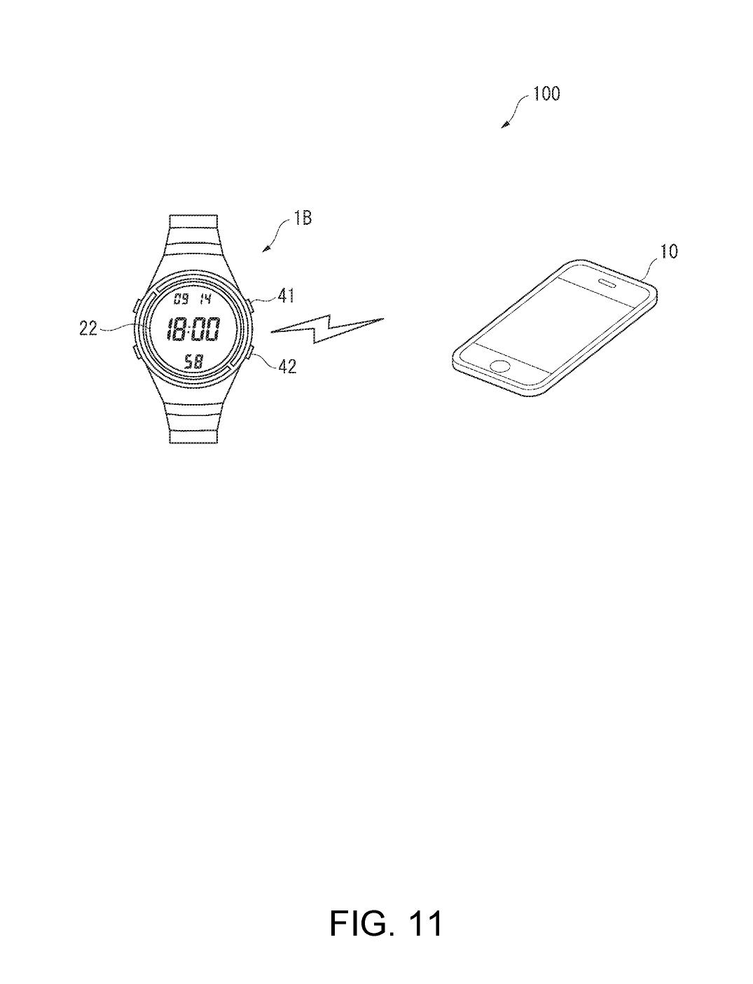

FIG. 11 is a schematic diagram showing a biological information processing system 100 according to the embodiment.

The biological information processing system 100 includes a biological information detection apparatus 1B and an information processing apparatus 10. The biological information detection apparatus 1B has the same exterior and substantially the same function as those of the above described biological information processing apparatus 1A. Further, the information processing apparatus 10 includes e.g. a smartphone (multifunctional portable telephone), a tablet, a PC (Personal Computer), or the like. These biological information detection apparatus 1B and information processing apparatus 10 are communicably connected to each other via Bluetooth or the like.

### Configuration of Biological Information Detection Apparatus

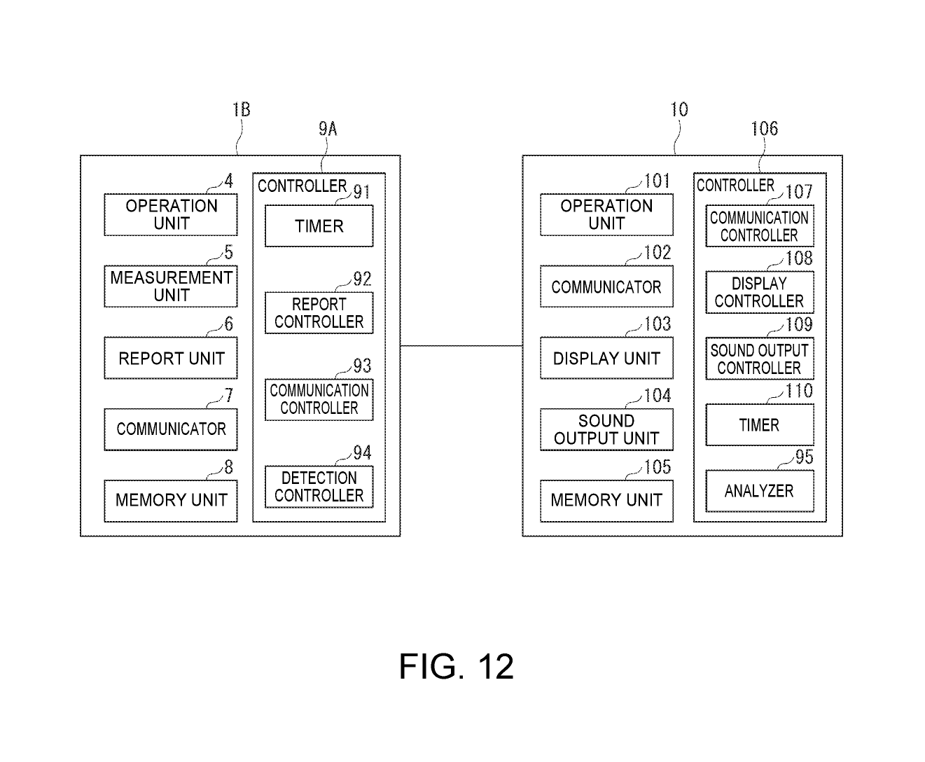

FIG. 12 is a block diagram showing a configuration of the biological information detection apparatus 1B and the information processing apparatus 10 according to the embodiment.

As shown in FIG. 12, the biological information detection apparatus 1B includes the operation unit 4, the measurement unit 5, the report unit 6, the communicator 7, the memory unit 8, and a controller 9A in place of the controller 9. The controller 9A includes the timer 91, the report controller 92, the communication controller 93, and the detection controller 94. That is, in the embodiment, the biological information detection apparatus 1B does not include the analyzer 95.

Further, the pulse wave signal and the electrocardiogram signal of the user measured by the measurement unit 5 of the biological information detection apparatus 1B are transmitted to the information processing apparatus 10 via the communicator 7 under the control of the communication controller 93. That is, the communicator 7 corresponds to a transmitter according to the invention.

Note that, in the embodiment, the pulse wave signal and the electrocardiogram signal are stored in the detection information memory part 82 of the memory unit 8 and transmitted to the information processing apparatus 10 via the communicator 7. Further, though the details will be described later, the pulsebeat confirmation processing by the analyzer 95 is executed in the information processing apparatus 10 and the pulse rate with high reliability after execution of the confirmation processing is received via the communicator 7. Thus acquired pulse rate is displayed on the display part 61 under the control of the report controller 92.

### Configuration of Information Processing Apparatus

As shown in FIG. 12, the information processing apparatus 10 includes an operation unit 101, a communicator 102, a display unit 103, a sound output unit 104, a memory unit 105, and a controller 106.

### Configuration of Operation Unit

The operation unit 101 receives an input operation by a user and outputs operation information in response to the input operation to the controller 106. The operation unit 101 may include e.g. physical keys and touch panels provided on a casing of the information processing apparatus 10, and a keyboard and a pointing device connected to the information processing apparatus 10 in wired or wireless connection.

### Configuration of Communicator

The communicator 102 includes a first communication module communicable with an external apparatus including the biological information detection apparatus 1B and a second communication module communicable with a server (not shown) on a network such as the Internet, and communicates with the external apparatus and the server under the control of the controller 106. Note that, in the case where each of the external apparatus and the server and the communicator 102 can communicate in the same communication system, the communicator 102 may include only one of the first communication module and the second communication module. In the case where the communication with the server is unnecessary, the second communication module may not necessarily be provided.

### Configurations of Display Unit and Sound Output Unit

The display unit 103 may include various display panels of e.g. liquid crystal, organic EL (Electro-Luminescence), electrophoresis, or the like, and displays screens for displaying the pulse rate etc. generated by the controller 106. Further, the display unit 103 displays screens prompting the user to measure the heart rate etc. under the control of the display controller 108, which will be described later.

The sound output unit 104 includes a speaker and outputs sound according to sound information input from the controller 106. For example, in the case where it is impossible for the controller 106 to specify the pulse rate or the like, the sound output unit 104 outputs sound according to information for prompting the user to measure the heart rate.

### Configuration of Memory Unit

The memory unit 105 includes a memory device such as an SSD (Solid State Drive), an HDD (Hard Disk Drive), or a flash memory, and stores programs and data necessary for the operation of the information processing apparatus 10. As the programs, the memory unit 105 stores an OS for controlling the information processing apparatus 10 and applications for execution of the same functions as those of the analyzer 95.

Further, the memory unit 105 stores various kinds of information received from the biological information detection apparatus 1B.

### Configuration of Controller

As shown in FIG. 12, the controller 106 includes a communication controller 107, a display controller 108, a sound output controller 109, a timer 110, and the analyzer 95.

The communication controller 107 controls the communicator 102 to communicate with the external apparatus and the server.

The display controller 108 allows the display unit 103 to display a screen for displaying the pulse rate of the user, a screen prompting user to measure electrocardiogram, and execution screens of the other applications and the OS (execution screens generated by the other configurations).

The sound output controller 109 outputs sound information of sound to be output when the OS and the applications are executed to the sound output unit 104.

The timer 110 times current date and time.

### Configuration of Analyzer

The analyzer 95 has the same function to that of the analyzer 95 of the controller 9 of the biological information processing apparatus 1A. Accordingly, in the information processing apparatus 10, when the pulse wave signal and the electrocardiogram signal of the user are received via the communicator 102, the above described pulsebeat confirmation processing and pulsebeat fixing processing (steps S11 to S13, steps S131 to S138, and steps S381 to S384) are executed in the analyzer 95.

Then, the pulse rate with high reliability calculated by execution of the pulsebeat confirmation processing and the pulsebeat fixing processing is displayed on the display unit 103 under the control of the display controller 108. Further, the pulse rate is transmitted to the biological information detection apparatus 1B via the communicator 102 received by the communicator 7 of the biological information detection apparatus 1B, and the received pulse rate is displayed on the display part 61 under the control of the communication controller 107.

Furthermore, at step S137, when the user is prompted to measure electrocardiogram, the communication controller 107 transmits the screen prompting the electrocardiogram measurement via the communicator 102 and allows the display part 61 of the biological information detection apparatus 1B to display the screen. Concurrently, the display controller 108 allows the display unit 103 of the information processing apparatus 10 to display the screen.

### Advantages of Second Embodiment

In the above described biological information processing system 100 according to the embodiment, besides the same advantages as those of the biological information processing apparatus 1A, the following advantages are obtained.

The biological information detection apparatus 1B does not execute the pulsebeat confirmation processing and the pulsebeat fixing processing, and the processing in the biological information detection apparatus 1B may be simplified.

Further, the pulse rate of the user is displayed on both the display unit 103 of the information processing apparatus 10 and the display part 61 of the biological information detection apparatus 1B, and thereby, the convenience of the user may be improved.

Furthermore, the screen prompting measurement of the heart rate is displayed on both the display unit 103 and the display part 61, and the possibility of recognition of the screen by the user is higher. Thereby, the possibility of the measurement of the heart rate by the user is higher, the elapsed time from the second time may be further shortened, and thereby, the possibility of calculation of the pulse rate with high reliability may be made higher.

### Modifications of Embodiments

The invention is not limited to the above described respective embodiments, and the invention includes modifications and improvements within the range in which the purpose of the invention may be achieved.

In the above described respective embodiments, if it is impossible to specify the pulse rate of the user, in other words, if the SN ratio is lower than the predetermined value, the pulse wave processor 98 calculates the pulse rate of the user based on the heart rate and the pulse wave signal of the user. However, the invention is not limited to that. For example, the above described pulsebeat confirmation processing may be executed when the body motion of the user (exercise intensity of the user) does not reach predetermined exercise intensity.

In the above described respective embodiments, if the difference between the first time and the second time is the predetermined period (e.g. within 60 seconds), in other words, the heart rate most recently stored is stored in the detection information memory part 82, the pulsebeat confirmation processing is executed. However, the invention is not limited to that. For example, even in the case where there is no most recently stored heart rate, if there is a heart rate stored in the past, the processing may be executed based on the heart rate. Or, in the case where there is no most recently stored heart rate, a heart rate based on the pulse rate most recently specified by the pulsebeat specification part 983 may be obtained and the processing may be executed based on the heart rate.

In the above described respective embodiments, the frequency range is set to be wider according to the elapsed time from the second time when the electrocardiogram signal is acquired. However, the invention is not limited to that. For example, a fixed acceptable range may be set regardless of the elapsed time from the second time.

In the above described respective embodiments, if the difference exceeds the predetermined period, in other words, if there is no most recently stored heart rate, the screen prompting the user to measure the electrocardiogram is displayed on at least one of the display part 61 and the display unit 103. However, the invention is not limited to that. For example, the screen may not necessarily be displayed or sound prompting the user to measure the electrocardiogram may be output in place of the display. Or, in place of or in addition to that, at least one of the biological information processing apparatus 1A, the biological information detection apparatus 1B, and the information processing apparatus 10 may be vibrated.

In the above described respective embodiments, the pitch calculator 972 is provided. However, the invention is not limited to that. For example, the pitch calculator 972 may not necessarily be provided. Or, in place of the pitch calculator 972, an exercise intensity detector that detects exercise intensity of the user based on the pulse rate fixed by the pulse wave processor 98 and the acceleration signal of the user may be provided.

In the above described respective embodiments, the time when the electrocardiogram is acquired with the heart rate by the heartbeat acquisition part 961 is stored in the detection information memory part 82. However, the invention is not limited to that. For example, the time when the electrocardiogram signal is detected may be stored with the heart rate.

In the above described second embodiment, the screen prompting the user to measure the electrocardiogram is displayed on both the display part 61 of the biological information detection apparatus 1B and the display unit 103 of the information processing apparatus 10. However, the invention is not limited to that. For example, the screen may be displayed on one of the display part 61 and the display unit 103.

Further, in the above described second embodiment, in addition to the display unit 103, the pulse rate on which the pulsebeat confirmation processing and the pulsebeat fixing processing have been executed is also displayed on the display part 61 of the biological information detection apparatus 1B. However, the invention is not limited to that. For example, the screen may be displayed on one of the display part 61 and the display unit 103.

In the above described second embodiment, the controller 106 of the information processing apparatus 10 includes the analyzer 95. However, the invention is not limited to that. For example, the analyzer 95 may be provided in a server or the like, the pulse wave signal and the electrocardiogram signal acquired from the biological information detection apparatus 1B by the information processing apparatus 10 are transmitted to the server, and the pulsebeat confirmation processing and the pulsebeat fixing processing may be executed in the server. In this case, the information processing apparatus 10 may acquire the results of the pulsebeat confirmation processing and the pulsebeat fixing processing executed by the server and transmit the results to the biological information detection apparatus 1B.

In the above described respective embodiments, the front surface-side electrode 541 and the back surface-side electrode 542 are provided. However, the invention is not limited to that. For example, the respective electrodes 541 and 542 may be provided on one of them.

In the above described respective embodiments, the analyzer 95 calculates the pulse rate as the biological information of the user. However, the invention is not limited to that. For example, blood pressure, blood sugar level, or the like of the user may be calculated as the biological information.

What is claimed is:

1. A biological information processing apparatus comprising:
a pulse wave acquisition part that acquires a pulse wave signal of a user;
an electrocardiogram acquisition part that acquires an electrocardiogram signal of the user; and
a processor that calculates biological information of the user based on the pulse wave signal,
wherein the processor calculates the biological information of the user based on a heart rate calculated from the electrocardiogram signal and the pulse wave signal if the pulse wave signal does not satisfy a predetermined condition.

2. The biological information processing apparatus according to claim 1, wherein the predetermined condition includes an index of at least one of an SN ratio of the pulse wave signal and body motion of the user.

3. The biological information processing apparatus according to claim 1, wherein the processor derives frequency information based on the heart rate and analyzes the pulse wave signal based on the frequency information.

4. The biological information processing apparatus according to claim 3, wherein the frequency information is information estimated based on the heart rate and representing a frequency range in which the pulse wave signal exists.

5. The biological information processing apparatus according to claim 4, further comprising a timer that times a period,
wherein the processor compares a first time when the pulse wave signal is acquired and a second time when the electrocardiogram signal is acquired, and, if a difference between the first time and the second time is within a predetermined period, analyzes the pulse wave signal based on the heart rate calculated from the electrocardiogram signal according to a comparison result and calculates the biological information.

6. The biological information processing apparatus according to claim 5, wherein the frequency range is wider according to an elapsed time from the second time when the electrocardiogram signal is acquired.

7. The biological information processing apparatus according to claim 5, further comprising a report unit that prompts the user to acquire the electrocardiogram signal if the difference between the first time and the second time exceeds the predetermined period.

8. A biological information processing system comprising:
a detection apparatus having
a pulse wave detector that detects a pulse wave signal of the user,
an electrocardiogram detector that detects an electrocardiogram signal of the user, and
a transmitter that transmits the pulse wave signal and the electrocardiogram signal; and
the biological information processing apparatus according to claim 1,
wherein the biological information processing apparatus includes a receiver that receives the pulse wave signal and the electrocardiogram signal.

9. The biological information processing system according to claim 8, wherein the detection apparatus includes a casing that houses the pulse wave detector, the electrocardiogram detector, and the transmitter,
the electrocardiogram detector includes
a first surface-side electrode provided on a first surface in the casing, and
a second surface-side electrode provided on a second surface different from the first surface in the casing.

10. A biological information processing method comprising:
acquiring a pulse wave signal of a user;
acquiring an electrocardiogram signal of the user; and
a processing of calculating biological information of the user based on the pulse wave signal,
wherein the processing calculates the biological information of the user based on a heart rate calculated from the electrocardiogram signal and the pulse wave signal if the pulse wave signal does not satisfy a predetermined condition.

11. A biological information processing program executed by a computer and allowing the computer to function as a biological information processing apparatus, comprising:
a pulse wave acquisition part that acquires a pulse wave signal of a user;
an electrocardiogram acquisition part that acquires an electrocardiogram signal of the user; and
a processor that calculates biological information of the user based on the pulse wave signal,
wherein the processor calculates the biological information of the user based on a heart rate calculated from the electrocardiogram signal and the pulse wave signal if the pulse wave signal does not satisfy a predetermined condition.

12. A biological information processing system comprising:
a detection apparatus having
a pulse wave detector that detects a pulse wave signal of the user,
an electrocardiogram detector that detects an electrocardiogram signal of the user, and
a transmitter that transmits the pulse wave signal and the electrocardiogram signal; and
the biological information processing apparatus according to claim 2,
wherein the biological information processing apparatus includes a receiver that receives the pulse wave signal and the electrocardiogram signal.

13. A biological information processing system comprising:
a detection apparatus having
a pulse wave detector that detects a pulse wave signal of the user,
an electrocardiogram detector that detects an electrocardiogram signal of the user, and
a transmitter that transmits the pulse wave signal and the electrocardiogram signal; and
the biological information processing apparatus according to claim 3,
wherein the biological information processing apparatus includes a receiver that receives the pulse wave signal and the electrocardiogram signal.

14. A biological information processing system comprising:
a detection apparatus having
a pulse wave detector that detects a pulse wave signal of the user,
an electrocardiogram detector that detects an electrocardiogram signal of the user, and
a transmitter that transmits the pulse wave signal and the electrocardiogram signal; and
the biological information processing apparatus according to claim 4,
wherein the biological information processing apparatus includes a receiver that receives the pulse wave signal and the electrocardiogram signal.

15. A biological information processing system comprising:
a detection apparatus having
a pulse wave detector that detects a pulse wave signal of the user,
an electrocardiogram detector that detects an electrocardiogram signal of the user, and
a transmitter that transmits the pulse wave signal and the electrocardiogram signal; and
the biological information processing apparatus according to claim 5,
wherein the biological information processing apparatus includes a receiver that receives the pulse wave signal and the electrocardiogram signal.

16. A biological information processing system comprising:
a detection apparatus having
a pulse wave detector that detects a pulse wave signal of the user,
an electrocardiogram detector that detects an electrocardiogram signal of the user, and
a transmitter that transmits the pulse wave signal and the electrocardiogram signal; and
the biological information processing apparatus according to claim 6,
wherein the biological information processing apparatus includes a receiver that receives the pulse wave signal and the electrocardiogram signal.

17. A biological information processing system comprising:
a detection apparatus having
a pulse wave detector that detects a pulse wave signal of the user,
an electrocardiogram detector that detects an electrocardiogram signal of the user, and
a transmitter that transmits the pulse wave signal and the electrocardiogram signal; and
the biological information processing apparatus according to claim 7,
wherein the biological information processing apparatus includes a receiver that receives the pulse wave signal and the electrocardiogram signal.
